(19) United States
(12) Patent Application Publication
(10) Pub. No.: US 2002/0058339 A1
(43) Pub. Date: May 16, 2002

(54) Carrier for co-culturing a fertilized ovum of an animal and method of culturing a fertilized ovum of an animal using the carrier

(75) Inventors: Toshiaki Takezawa, Tsukuba-shi (JP); Kei Imai, Tsukuba-shi (JP); Toru Takahashi, Tsukuba-shi (JP); Kazuyoshi Hashizume, Tokyo (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Bio-oriented Technology Research Advancement Institution, Omiya-shi (JP)

(21) Appl. No.: 09/775,648

(22) Filed: Feb. 5, 2001

(30) Foreign Application Priority Data
Nov. 15, 2000 (JP) 2000-347924

(51) Int. Cl. C12N 15/00; C12M 1/22; C12M 3/00; C12N 5/00; C12N 5/02

(52) U.S. Cl. 435/404; 435/305.1; 800/21

(57) ABSTRACT

There is provided a carrier for co-culturing a fertilized ovum of an animal by which behavior of the fertilized ovum of an animal can be easily observed in a culture system and by which adhesion and three-dimensional growth of the fertilized ovum become possible at first, as well as there is provided a method of culturing the fertilized ovum of an animal using the co-culturing carrier. There is provided a carrier for co-culturing a fertilized ovum of an animal composed of a cell incorporated type three-dimensionally reconstructed tissue for co-culturing the fertilized ovum of an animal to induce adhesion and three-dimensional growth, of the fertilized ovum as well as there is provided a method of culturing the fertilized ovum of an animal characterized in that the co-culturing carrier is introduced into a culture vessel to culture the fertilized ovum of an animal.

### FIELD OF THE INVENTION

The present invention relates to a carrier for co-culturing a fertilized ovum of an animal and a method of culturing the fertilized ovum of an animal using the carrier. More precisely, the invention relates to a co-culturing carrier composed of a cell incorporated type three-dimensionally reconstructed tissue for inducing adhesion and three-dimensional growth of the fertilized ovum of an animal in a culture system and a method of culturing the fertilized ovum of an animal using the carrier.

According to the invention, the fertilized ovum of an animal can be grown three-dimensionally in a culture system, and thus the invention is useful for elucidation of the differences between the three-dimensional growth of the fertilized ovum in an in vitro culture system and the development of the early embryo from the fertilized ovum implanted in vivo, evaluation of teratogenic materials, or grafting of an embryo initially developed from the fertilized ovum, etc.

### BACKGROUND OF THE INVENTION

Hitherto, there has been established an assisted reproductive technology (ART) not only in a veterinary field but also in a human sterility treatment, wherein a spermatozoon and an ovum are fertilized in vitro in a culture system to prepare a fertilized ovum (a zygote), then the fertilized ovum can be cultured via cleavage, morula and blastocyst stages to a hatching-blastocyst stage, a late blastula stage wherein a zona pellucida is denatured and disappeared, and the fertilized ovum at the stages from cleavage to blastula stage is transplanted in an uterus to obtain a baby.

Also, a fertilized ovum (a late blastula) is implanted on an endometrium in vivo and an inner cell mass (an embryoblast) grows to a development stage of early embryo including a gastrula forming process, which proceeds to a three-layer embryonic disc. There is, however, not yet any report about such possible growth in a culture system.

Namely, in the culture systems hitherto, even if a fertilized ovum (a late blastula) is cultured continuously, only monolayer cells are proliferated two-dimensionally and any three-dimensional architecture having an early embryo-like structure such that a gastrula or a neurula is produced has not been yet prepared.

On one hand, the basic technology of tissue engineering for reconstruction of a tissue from both cultured cells and their scaffold(s) (a culture carrier(s)) has proceeded eminently for these 10 years centering around Europe and America. As to organs having relatively simple constructions, the basic reconstruction method has been established [Ferber, D., Science 284, 422-425, (1999)].

Hitherto, in order to construct a tissue by assembling cells and extracellular matrix components three-dimensionally, there have been developed carriers having various forms from many materials.

We, the inventors, have established the basic technology of tissue engineering with utilizing a mesh network such as cotton gauze as a support (Japanese Patent Application Laid-open No. Hei 7-298876 and Japanese Patent No. 3081130).

Further, the inventors have already established a novel organ engineering method of reconstructing an organ-like construct (an organoid) by subjecting continuous three-step perfusion on an organ to remodel the organ into a culture version organoid without separating the majority of constructive cells in the objective organ (Japanese Patent Application Laid-open No. Hei 11-164684).

Further, as to a reconstructing technology of an endometrium, it has been reported that the endometrial epithelial cells reconstructs a uterine gland-like structure by co-culturing human endometrial epithelial cells and stromal cells in a collagen gel [Akoum, A., et al., J. Reprod. Med., 41, 555-561, (1996)].

Further, for rabbit, there is a report to culture endometrial epithelial cells on a matrigel, a reconstituted basement membrane, and thereafter to place a blastocyst just before implantation thereon for co-culturing. Although there is described that cell fusion of a trophoblast (cytotrophoblast) with the endometrial epithelial cells occurred at 48 hours after co-culturing [Tominaga Tosirou, Nihon Sanfujinka Gakkai Zasshi, 48, 591-603, (1996)], there is no description that the cells derived from the blastocyst grow to form a three-dimensional architecture having an early embryo-like structure such that a gastrula or a neurula is formed.

Namely, there is not yet any report of a culture carrier or a co-culturing carrier on which a fertilized ovum of an animal is cultured to induce three-dimensional growth.

### SUMMARY OF THE INVENTION

An object of the invention is to provide a carrier for co-culturing a fertilized ovum of an animal in which behavior of the fertilized ovum of an animal can be easily observed in a culture system and by which adhesion and three-dimensional growth of the fertilized ovum become possible at first.

Further, another object of the invention is to provide a method of culturing the fertilized ovum of an animal, in which the fertilized ovum of an animal can be grown three-dimensionally by culturing the fertilized ovum of an animal using the co-culturing carrier and in which elucidation of the differences between the three-dimensional growth of the fertilized ovum in an in vitro culture system and the development of the early embryo from the fertilized ovum implanted in vivo, evaluation of teratogenic materials, or grafting of an early embryo developed from the fertilized ovum, etc. become possible.

In order to develop a carrier for co-culturing a fertilized ovum of an animal and a culturing method of a fertilized ovum of an animal in which behavior of the fertilized ovum of an animal can be easily observed in a culture system and in which adhesion and three-dimensional growth of the fertilized ovum become possible, the inventors have studied eagerly and found that a carrier for co-culturing a fertilized ovum of an animal composed of a cell incorporated type three-dimensionally reconstructed tissue in which cells are beforehand incorporated in a culture carrier makes adhesion and three-dimensional growth of the fertilized ovum possible to complete the present invention based on this founding.

Namely, according to the first aspect of the invention, there is provided a carrier for co-culturing a fertilized ovum of an animal comprising a cell incorporated type three-dimensionally reconstructed tissue for co-culturing the fertilized ovum of an animal to induce adhesion and three-dimensional growth of the fertilized ovum.

Further, according to the tenth aspect of the invention, there is provided a method of culturing a fertilized ovum of an animal, characterized in that any co-culturing carrier as described in the first to ninth aspects of the invention is introduced into a culture vessel to culture the fertilized ovum of an animal.

### BRIEF DESCRIPTION OF THE DRAWINGS

In the accompanying drawings:

FIG. 1 is a photograph for gross appearance of co-culturing carriers composed of a cell incorporated type three-dimensionally reconstructed tissue containing gauze or not containing gauze at the fifth day of culturing;

FIG. 2 is a phase-contrast microphotograph of the co-culturing carrier composed of a cell incorporated type three-dimensionally reconstructed tissue not containing gauze at the fifth day of culturing;

FIG. 3 is an optical microphotograph of a hematoxylin-eosin stained section of the co-culturing carrier composed of a cell incorporated type three-dimensionally reconstructed tissue not containing gauze at the seventh day of culturing;

FIG. 4 is an optical microphotograph (at low magnification) of a hematoxylin-eosin stained section of the co-culturing carrier composed of a cell incorporated type three-dimensionally reconstructed tissue not containing gauze at the seventh day of culturing;

FIG. 5 is a phase-contrast microphotograph of the co-culturing carrier composed of a cell incorporated type three-dimensionally reconstructed tissue containing gauze at the seventh day of culturing;

FIG. 6 is a phase-contrast microphotograph at the first day after a fertilized ovum was co-cultured on the co-culturing carrier containing gauze;

FIG. 7 is a phase-contrast microphotograph at the second day after a fertilized ovum was co-cultured on the co-culturing carrier containing gauze;

FIG. 8 is a phase-contrast microphotograph at the sixth day after a fertilized ovum was co-cultured on the co-culturing carrier containing gauze;

FIG. 9 is a phase-contrast microphotograph at the twelfth day after a fertilized ovum was co-cultured on the co-culturing carrier containing gauze;

FIG. 10 is a phase-contrast microphotograph (at high magnification) at the twelfth day after a fertilized ovum was co-cultured on the co-culturing carrier containing gauze;

FIG. 11 is an optical microphotograph (at high magnification) of a hematoxylin-eosin stained section of the co-culturing carrier by which a fertilized ovum was co-cultured for 12 days;

FIG. 12 is an optical microphotograph (at low magnification) of a hematoxylin-eosin stained section of the co-culturing carrier by which a fertilized ovum was co-cultured for 12 days;

FIG. 13 is an optical microphotograph (at high magnification) for another site of a hematoxylin-eosin stained section of the co-culturing carrier by which a fertilized ovum was co-cultured for 12 days; and

FIG. 14 is an optical microphotograph (at low magnification) for another site of a hematoxylin-eosin stained section of the co-culturing carrier by which a fertilized ovum was co-cultured for 12 days.

### DETAILED DESCRIPTION OF THE INVENTION

At first, a carrier for co-culturing a fertilized ovum of an animal according to the first aspect of the invention is illustrated.

The carrier for co-culturing the fertilized ovum of an animal according to the first aspect of the invention is the carrier for co-culturing the fertilized ovum of an animal to induce adhesion and three-dimensional growth of the fertilized ovum, wherein the carrier is composed of the cell incorporated type three-dimensionally reconstructed tissue.

The fertilized ovum of an animal, which is an object of the carrier for co-culturing the fertilized ovum of an animal according to the first aspect of the invention, may be any fertilized ovum derived from mammal or from other animal.

As mammal, there may be mentioned human beings, monkey, bovine, sheep, goat, baboon, pig, dog, guinea pig, rat and mouse etc.

Further, the fertilized ovum used in culturing may be any stage of a zygote, cleavage, morula or blastocyst stage, but one grown to a blastocyst stage is preferable as an implantation model.

Further, in the culture system according to the first aspect of the invention, any ovum in a life cycle other than a fertilized ovum, namely an ovum cell before fertilization such as an ovum in follicule and an ovulated ovum or a fertilizing ovum, may be used.

The carrier for co-culturing the fertilized ovum of an animal according to the first aspect of the invention is the carrier for co-culturing the above mentioned fertilized ovum of an animal to induce adhesion and three-dimensional growth of the fertilized ovum.

Namely, in addition that the carrier for co-culturing the fertilized ovum of an animal according to the first aspect of the invention is suitable for adhesion of the fertilized ovum, not only it cultures the fertilized ovum (blastocyte) as hitherto to proliferate monolayer cells two-dimensionally but also it can prepare a three-dimensional architecture derived from the fertilized ovum.

Such characteristics are based on the following construction of the carrier for co-culturing the fertilized ovum of an animal according to the first aspect of the invention.

The carrier for co-culturing the fertilized ovum of an animal according to the first aspect of the invention is composed of the cell incorporated type three-dimensionally reconstructed tissue.

Herein, the cell incorporated type three-dimensionally reconstructed tissue is one to become a scaffold for growing a three-dimensional tissue derived from the fertilized ovum.

The cells to be incorporated in the cell incorporated type three-dimensionally reconstructed tissue are cells derived from an animal that is homogeneous or heterogeneneous to the fertilized ovum as is described in the fourth aspect of the invention.

Further, the cells may be primary cultured cells, strained cells or cells transfected with an exogeneous gene(s). Further, the cells may be one kind or two or more kinds.

In particular, in the case of preparing the cell incorporated type three-dimensionally reconstructed tissue as an implantation model of the fertilized ovum into an endometrium, the cells to be incorporated in the cell incorporated type three-dimensionally reconstructed tissue are preferably cells derived from an endometrium, particularly endometrial epithelial cells and stromal cells as is described in the fifth aspect of the invention.

Similarly, as the cells to be incorporated in the cell incorporated type three-dimensionally reconstructed tissue, cells derived from an ovary or cells derived from an uterine tube may be used for reflecting an in vivo environment about a life cycle of the ovum to be cultured.

Further, as is described in the sixth aspect of the invention, the ability of the cell division can be lost by pretreating the cells to be incorporated with mitomycin C, thus the three-dimensionally reconstructed tissue may be obtained which can accelerate three-dimensional growth of the fertilized ovum.

Such cell incorporated type three-dimensionally reconstructed tissue is reconstructed from any of cells, tissues or organs derived from animal, and it contains at least one kind of cells as is described in the second aspect of the invention.

That is, for example, it can be obtained by culturing the above-mentioned cells with a culture medium.

The culture medium is not limited particularly if it has an ability of culturing cells, and, for example, Dulbecco's modified Eagle (DME)/F12 medium (containing 10% of heat-inactivated fetal bovine serum, 10 mM HEPES, 100 units/mL of penicillin, 100 μg/mL of streptomycin) etc. may be preferably used.

The cell incorporated type three-dimensionally reconstructed tissue preferably contains an extracellular matrix component and/or a mesh network as is described in the third aspect of the invention. By containing them, liquid permeability of the culture medium is improved to culture the incorporated cells effectively and to provide tension on the incorporated cells, whereby three-dimensional growth of the fertilized ovum can be proceeded in a condition more close to a living body.

Herein, the extracellular matrix component is referred to each component of extracellular matrices which act to support and adhere the cells in a living body, and there may be specifically mentioned, for example, collagen, fibronectin, vitronectin, laminin, proteoglycan, glycosaminoglycan, etc.

The extracellular matrix component(s) may be a component(s) derived from an identical animal with the cells to be incorporated in the cell incorporated type three-dimensionally reconstructed tissue or may be a component(s) derived from a different animal.

Further, as is described in the seventh aspect of the invention, the extracellular matrix component is preferably gelated.

For example, as a gel of the extracellular matrix component, there may be mentioned a collagen gel or a matrigel, etc.

Then, the mesh network is referred to a fibrous mass having such an opening to form a spatial shape for three-dimensional culturing, and as is described in the eighth aspect of the invention, there may be mentioned natural or synthetic threads and/or woven masses thereof.

As the mesh network, there may be mentioned the mesh network of natural threads such as cotton or silk, the mesh network of synthetic threads such as nylon, acryl or polyester, and the mesh network consisting of woven masses thereof. A criterion of a thread thickness is about 10-100 μm in diameter, and plural kinds of threads may be used in combination, if appropriate.

As the mesh network, there may be mentioned specifically cotton gauze such as sterilized gauze type III (K-Pine, made by Kawamoto Hotai Zairyo Co. Ltd.).

The physical form of the mesh network is not particularly limited if it can form a spatial shape for three-dimensional culturing, and the form may be selected appropriately, taking account of objective cells and culture conditions thereof.

Specifically, the size of the opening in the mesh network is within a range of 10-1000 μm, preferably 20-400 μm.

Further, as to water absorption, natural threads and/or woven masses thereof have higher absorption than synthetic threads and/or woven masses thereof. Viewing this point, those suitable for the characteristics of the cells to be incorporated should be selected.

In the present invention, only one mesh network may be used, but mesh networks partially modified in their physical forms or properties such as an opening size may be used and also two or more mesh networks having different physical forms or properties such as an opening size may be used in combination, if appropriate.

Further, the mesh network is preferably bioabsorptive according to the ninth aspect of the invention. The bioabsorption is referred to a property to be absorbed and degraded in a living body. Since it can absorb the culture carrier in a living body, it is quite useful for transplantation, etc.

The cell incorporated type three-dimensionally reconstructed tissue preferably contains the above-mentioned extracellular matrix component or the mesh network, or both of them.

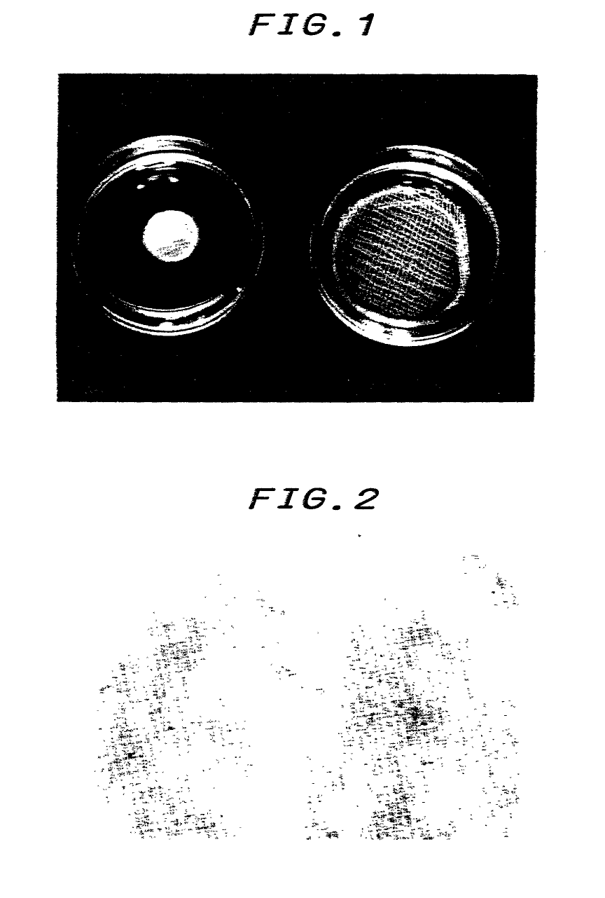

Herein, if the mesh network is not contained, it can become difficult to observe behavior of the fertilized ovum by a phase-contrast microscope, since the cell incorporated type three-dimensionally reconstructed tissue contracts and aggregates with the time lapsed although the cells are incorporated to make adhesion and three-dimensional growth of the fertilized ovum possible (see FIG. 1).

In contrast, by containing the mesh network, contraction of the cell incorporated type three-dimensionally reconstructed tissue is inhibited, so that behavior of the fertilized ovum can be preferably observed by means of a phase-contrast microscope, thus it is preferable.

The size and form of the cell incorporated type three-dimensionally reconstructed tissue may be any one if adhesion and three-dimensional growth of the fertilized ovum can be supported thereby and if behavior of the fertilized ovum under culture can be easily observed by means of a phase-contrast microscope, thus they may be any size and form which are suitable for inserting a culture dish with a diameter of 35 mm.

As described above, such cell incorporated type three-dimensionally reconstructed tissue is reconstructed from any of animal-derived cells, tissues or organs and contains at least one kind of cells according to the second aspect of the invention, and, for example, it can be obtained by culturing at least the above-mentioned cells with the culture medium.

As specific methods for preparation of the cell incorporated type three-dimensionally reconstructed tissue, there may be mentioned, for example, a culturing method of a multicellular spheroid (spheroid) using a culture substratum with a thermo-responsive polymer, a culturing method using a gel of an extracellular matrix component(s), a culturing method with utilizing a mesh network (Japanese Patent Application Laid-open No. Hei 7-298876, Japanese Patent No. 3081130) and an organ engineering method by means of continuous three-step perfusion (Japanese Patent Application Laid-open No. Hei 11-164684), etc.

In the case that the cell incorporated type three-dimensionally reconstructed tissue containing the extracellular matrix component and/or mesh network according to the third aspect of the invention is prepared, a culturing method using a gel of an extracellular matrix component(s) and a culturing method utilizing a mesh network may be utilized among the above-mentioned methods.

For example, according to a culturing method using a gel of an extracellular matrix component(s), cells to be incorporated are suspended in a culture medium and mixed with the sol-state extracellular matrix component, and thereafter the cell suspension is seeded in a culture dish and cultured to embed the cells in the gel. By removing the gel having the embedded cells from the culture dish and subjecting to floating culture, the cell incorporated type three-dimensionally reconstructed tissue can be obtained.

According to a culturing method utilizing a mesh network (Japanese Patent Application Laid-open No. Hei 7-298876, etc.), cells to be incorporated are suspended in a culture medium and this cell suspension is mixed with a sol-state extracellular matrix component, and thereafter the cell suspension is seeded in a culture dish containing a mesh network and cultured to embed the cells and the mesh network in the gel. By removing the collagen gel having both embedded cells and gauze thus obtained from the culture dish and subjecting to floating culture, the cell incorporated type three-dimensionally reconstructed tissue can be obtained.

The co-culturing carrier according to the first aspect of the invention which is composed of the above-mentioned cell incorporated type three-dimensionally reconstructed tissue can be used for culturing of a fertilized ovum.

The tenth aspect of the invention relates to culturing a fertilized ovum of an animal using the co-culturing carrier according to the first aspect of the invention, the co-culturing carrier composed of above described cell incorporated type three-dimensionally reconstructed tissue.

The tenth aspect of the invention relates to a culturing method of a fertilized ovum, characterized by introducing any co-culturing carrier as described in the first to ninth aspects of the invention into a culture vessel and culturing a fertilized ovum of an animal.

Namely, any co-culturing carrier as described in the first to ninth aspects of the invention is introduced into a culture vessel and a fertilized ovum is placed on the co-culturing carrier, and the fertilized ovum is cultured with feeding a culture medium to the fertilized ovum and the cells incorporated in the co-culturing carrier (cell incorporated type three-dimensionally reconstructed tissue).

As the culture medium, there may be used those used for preparing a cell incorporated type three-dimensionally reconstructed tissue. Such culture medium is changed every one to three day(s). The culturing temperature is 37.0-39.0° C. and the culturing period is about 1-60 day(s).

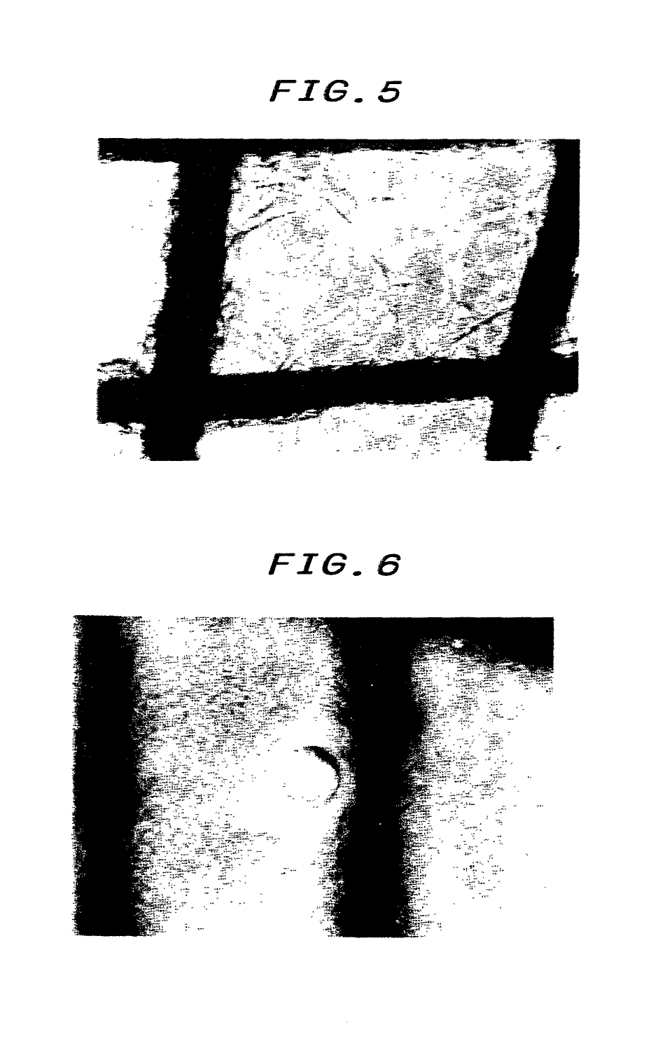

When the fertilized ovum is cultured by a culturing method according to the tenth aspect of the invention, behavior of the fertilized ovum during culturing can be observed by means of a phase-contrast microscope, etc. (see FIGS. 6-10) and also the cells derived from the fertilized ovum are moved around the fertilized ovum to make three-dimensional growth of the fertilized ovum possible finally (see FIGS. 11-14).

According to the first aspect of the invention, there is provided a carrier for co-culturing a fertilized ovum composed of a cell incorporated type three-dimensionally reconstructed tissue, and thus, behavior of the fertilized ovum of an animal in a culture system can be observed easily and adhesion and three-dimensional growth of the fertilized ovum become possible at first.

Further, by a culturing method according to the tenth aspect of the invention using the co-culturing carrier, a fertilized ovum of an animal can be grown three-dimensionally, and thus elucidation of the differences between the three-dimensional growth of the fertilized ovum in an in vitro culture system and the development of the early embryo from the fertilized ovum implanted in vivo, evaluation of teratogenic materials, or grafting of an early embryo developed from the fertilized ovum, etc. become possible.

### EXAMPLES

Preparation Example 1

(Preparation of a Co-culturing Carrier Composed of a Cell Incorporated Type Three-dimensionally Reconstructed Tissue)

Endometrial epithelial cells that had been subjected to primary culture from bovine uterus and thereafter to subcultures for several times (referred to epithelial cells, hereinafter) and endometrial stromal cells (referred to stromal cells, hereinafter) were suspended in culture medium (DME/F12 medium containing 10% of heat-inactivated fetal bovine serum, 10 mM HEPES, 100 units/mL of penicillin, 100 μg/mL of streptomycin) in such a way that final cell densities thereof were 8.8×10⁵/m and 6.8×10⁵/mL, respectively.

After equal amounts of the cell suspension and a 0.5% type-I collagen aqueous solution (CELLGEN I-AC, made by KOKEN Co. Ltd.) were mixed on ice, they were seeded in 2.0 mL portions into a hydrophobic culture dish (Falcon #351008) having a diameter of 35 mm made of polystylene and cultured for 1 hour in a humidified incubator at 37° C. in the presence of 5% CO₂/95% air to gelate collagen completely.

On the collagen gel having the final concentration of 0.25%, 2.0 mL of the culture medium was added. After culturing for further 1 hour, the collagen gel having the embedded cells was removed from the culture dish and subjected to floating culture. Thereafter, it was cultured for 7 days with the culture medium being changed every other day.

As the result, the gel was gradually contracted and aggregated to attain a gel diameter of 22 mm at the second day of culturing, 11 mm at the fifth day and 9 mm at the seventh day (see FIG. 1). FIG. 1 is a photograph for gross appearance of the co-culturing carrier composed of a cell incorporated type three-dimensionally reconstructed tissue at the fifth day, wherein one contains gauze (right side in the picture) and one does not contain gauze (left side in the picture).

A phase-contrast microphotograph of a gel not containing gauze at the fifth day of culturing is shown in FIG. 2. 10 mm in FIG. 2 corresponds to actual 140 μm.

Owing to gel contraction, as is clear from FIG. 2, it was difficult to observe the cell morphology in the gel by means of a phase-contrast microscope at the fifth day of culturing.

Thus, the gel at the seventh day of culturing was fixed with formalin to prepare paraffin sections according to the conventional way. The morphology inside of the gel was observed by means of hematoxylin-eosin staining.

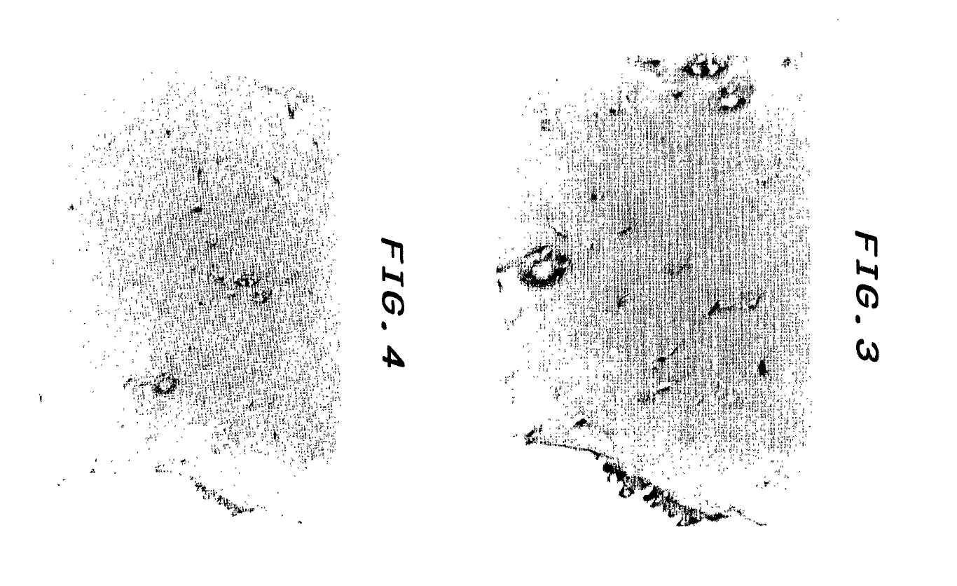

The state of the section after hematoxylin-eosin staining at the seventh day of culturing is shown in FIG. 3. 10 mm in FIG. 3 corresponds to actual 35 μm. Further, the same section after hematoxylin-eosin staining at the seventh day of culturing in FIG. 3, observed at the lower magnification, is shown in FIG. 4. 10 mm in FIG. 4 corresponds to actual 70 μm.

According to FIG. 3 and FIG. 4, it is clear that epithelial cells formed a monolayer cuboidal epithelium shape on a gel surface but formed a uterine gland-like structure inside of the gel, and thus it could be confirmed that the cells were incorporated in the gel.

From this, according to the Preparation Example 1, it was proved that the co-culturing carrier composed of the cell incorporated type three-dimensionally reconstructed tissue was obtained. It is presumed that the co-culturing carrier composed of the cell incorporated type three-dimensionally reconstructed tissue was prepared which can become an implantation model for a fertilized ovum into an endometrium when culturing a fertilized ovum.

### Preparation Example 2

### Preparation of a Co-culturing Carrier Composed of a Cell Incorporated Type Three-dimensionally Reconstructed Tissue Containing Gauze

After equal amounts of a cell suspension prepared similarly to Preparation Example 1 and a 0.5% type-I collagen aqueous solution (CELLGEN I-AC, made by KOKEN Co. Ltd.) were mixed on ice, they were seeded in 2.0 mL portions into a culture dish (Falcon #351008) similar to that used in Preparation Example 1 except that a sterilized gauze type III (K-Pine, made by Kawamoto Hotai Zairyo Co. Ltd.) cut circular to have its diameter of 34 mm was inserted, and cultured for 1 hour in a humidified incubator at 37° C. in the presence of 5% CO₂/95% air to gelate collagen completely.

On the collagen gel having the final concentration of 0.25%, 2.0 mL of culture medium was added. After culturing for further 1 hour, the collagen gel having both embedded cells and gauze was removed from the culture dish and subjected to floating culture. Thereafter, it was cultured for 7 days with the culture medium being changed every other day.

As the result, the gel diameter was 32 mm at the second day of culturing, 29 mm at the fifth day and 28 mm at the seventh day. In the co-culturing carrier containing gauze in the Preparation Example 2, contraction as seen in the Preparation Example 1 was not observed. Such phenomenon is recognized due to inhibition of gel contraction by means of gauze (see, FIG. 1).

A phase-contrast microphotograph of a collagen gel at the seventh day of culturing is shown in FIG. 5. 10 mm in FIG. 5 corresponds to actual 140 μm.

As shown in FIG. 5, since gel contraction was inhibited by means of gauze even at the seventh day of culturing, a cell morphology in a gel can be well observed by means of a phase-contrast microscope.

Further, according to FIG. 5, similar to the case in the Preparation Example 1, it is presumed that epithelial cells formed a monolayer cuboidal epithelium shape on a gel surface but formed a uterine gland-like structure inside of the gel.

From this, according to the Preparation Example 2, the co-culturing carrier composed of the cell incorporated type three-dimensionally reconstructed tissue was clearly obtained which can become an implantation model for a fertilized ovum into an endometrium.

### Example 1

### Culturing of a Fertilized Ovum on a Co-culturing Carrier Containing Gauze

On a co-culturing carrier composed of a cell incorporated type three-dimensionally reconstructed tissue containing gauze at the seventh day of culturing prepared in Preparation Example 2, a fertilized ovum (a blastocyst) at the seventh day after in vitro fertilization of bovine was co-cultured. Co-culture was carried out for 12 days with the culture medium being changed every other day.

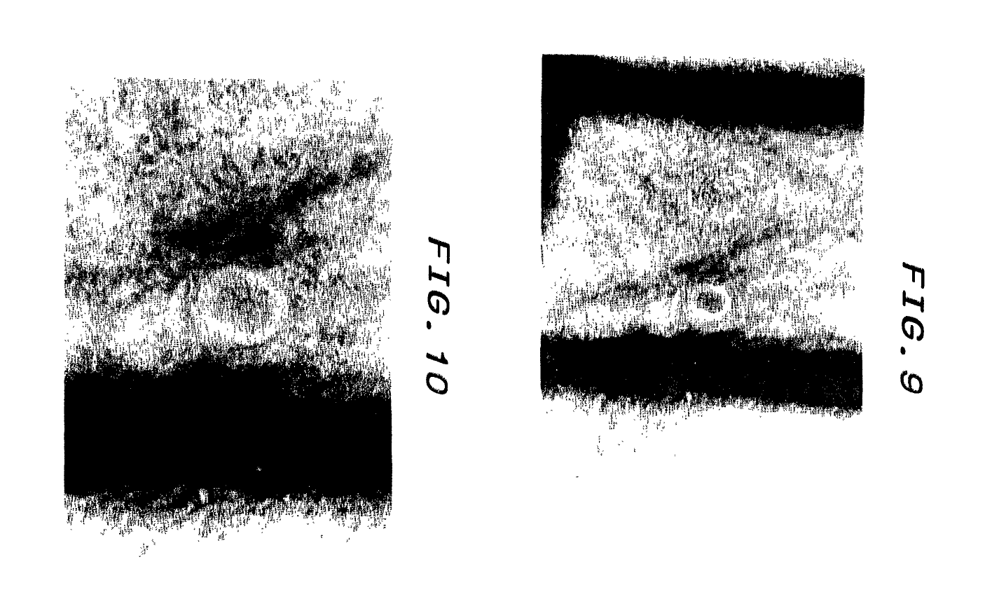

Behavior of the fertilized ovum was continuously observed by means of a phase-contrast microscope. In FIGS. 6-9, phase-contrast microphotograph at the first, the second, the sixth and the twelfth days after the fertilized ovum was co-cultured on the co-culturing carrier containing gauze are shown, respectively. 10 mm in respective pictures corresponds to actual 140 μm. Further, a phase-contrast microphotograph (at high magnification) at the twelfth day after the fertilized ovum was co-cultured on the co-culturing carrier same as in FIG. 9 is shown in FIG. 10. 10 mm in FIG. 10 corresponds to actual 70 μm.

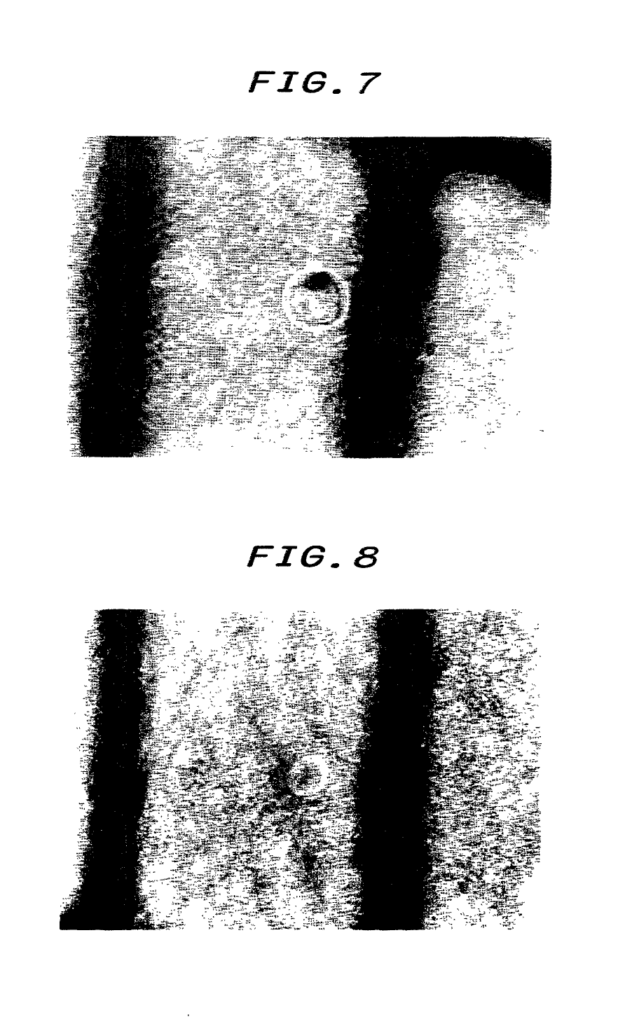

As the result, the fertilized ovum was not yet adhered to the co-culturing carrier at the first and the second days after co-culture of the fertilized ovum (see FIGS. 6, 7) and it was confirmed that the fertilized ovum was adhered to the co-culturing carrier at the sixth day after co-culture of the fertilized ovum (see FIG. 8). Further, a cell migration phenomenon considered to be derived from the fertilized ovum could be confirmed around the fertilized ovum at the twelfth day after co-culture (see FIGS. 9, 10).

Thus, the co-culturing carrier to which the fertilized ovum at the twelfth day after co-culture had been adhered was fixed with formalin to prepare resin sections for optical microscope according to the conventional method. The morphology inside of the co-culturing carrier was observed by means of hematoxylin-eosin staining.

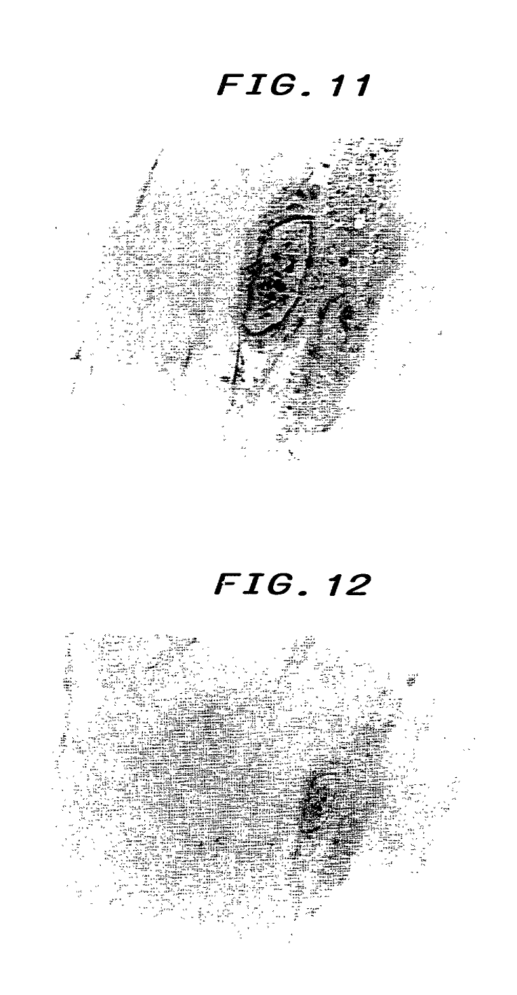

Optical microphotographs of hematoxylin-eosin staining sections of carriers for co-culturing after co-culturing of the fertilized ovum for 12 days are shown in FIG. 11 and FIG. 12. 10 mm in FIG. 11 and FIG. 12 corresponds to actual 35 μm and 70 μm, respectively.

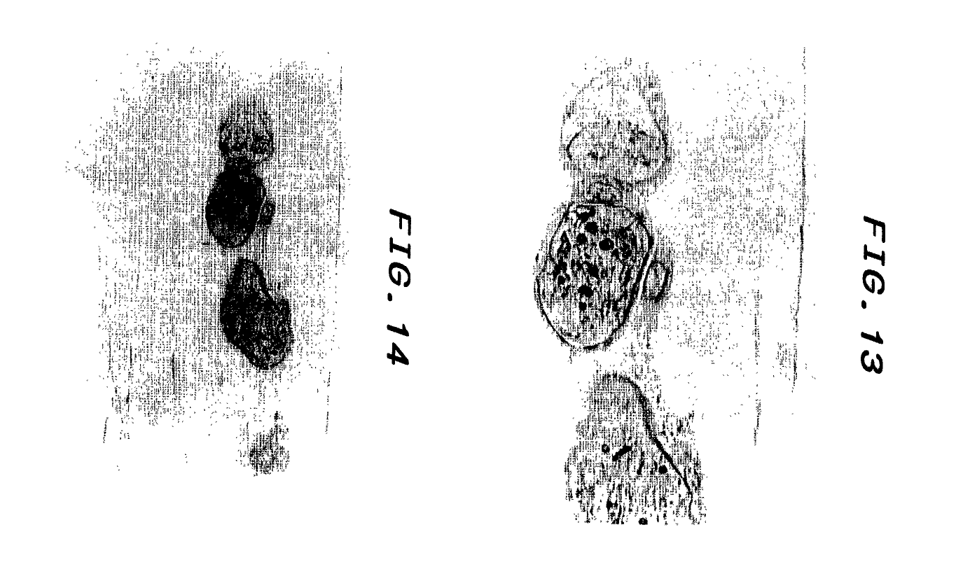

Further, similar optical microphotographs for other sites of the carriers for co-culturing are shown in FIG. 13 and FIG. 14. 10 mm in FIG. 13 and FIG. 14 corresponds to actual 35 μm and 70 μm, respectively.

According to FIGS. 11-14, it could be confirmed that not only monolayer cells were proliferated two-dimensionally but also a three-dimensional architecture considered to be due to growth of the fertilized ovum was formed by culturing the fertilized ovum.

From this, it was proved that behavior of a fertilized ovum can be easily observed by a phase-contrast microscope and also that adhesion and three-dimensional growth of the fertilized ovum become possible when a fertilized ovum is cultured on the co-culturing carrier of the present invention.

Of course, the invention is not limited to the above-mentioned Examples. There is no need to dwell upon various possible embodiments as to a life cycle state of an ovum as a culturing object, a composition of a culture medium, a culturing condition, cells used for preparation of a cell incorporated type three-dimensionally reconstructed tissue, and a kind of an extracellular matrix component and/or a mesh network.

What is claimed is:

1. A carrier for co-culturing a fertilized ovum of an animal comprising a cell incorporated type three-dimensionally reconstructed tissue for co-culturing the fertilized ovum of an animal to induce adhesion and three-dimensional growth of the fertilized ovum.

2. The co-culturing carrier according to claim 1, wherein the cell incorporated type three-dimensionally reconstructed tissue is reconstructed from any of cells, tissues or organs derived from animals and it contains at least one cell(s).

3. The co-culturing carrier according to claim 1 or 2, characterized in that the cell incorporated type three-dimensionally reconstructed tissue contains an extracellular matrix component(s) and/or a mesh network(s), in addition to the incorporated cells.

4. The co-culturing carrier according to any one of claims 1-3, characterized in that the cells to be incorporated in the cell incorporated type three-dimensionally reconstructed tissue are cells derived from an animal that is homogeneous or heterogeneous to the fertilized ovum.

5. The co-culturing carrier according to any one of claims 1-4, characterized in that the cells to be incorporated in the cell incorporated type three-dimensionally reconstructed tissue are cells derived from an endometrium.

6. The co-culturing carrier according to any one of claims 1-5, characterized in that the cell to be incorporated in the cell incorporated type three-dimensionally reconstructed tissue are pretreated with mitomycin C.

7. The co-culturing carrier according to claim 3, characterized in that the extracellular matrix component is gelated.

8. The co-culturing carrier according to claim 3, characterized in that the mesh network is composed of any natural or synthetic thread and/or woven mass thereof.

9. The co-culturing carrier according to claim 3 or 8, characterized in that the mesh network is bioabsorptive.

10. The method of culturing a fertilized ovum of an animal, characterized in that the co-culturing carrier according to any one of claims 1-9 is introduced into a culture vessel to culture the fertilized ovum of an animal.
